(19) United States
(12) Patent Application Publication
(10) Pub. No.: US 2005/0239076 A1
(43) Pub. Date: Oct. 27, 2005

(54) Analysis system

(76) Inventors: Caroline Garey, Cambridge (GB); Jodie Hadley, Ely (GB); Peter Swarbrick, Cambridge (GB)

Correspondence Address: STITES & HARBISON PLLC, 1199 NORTH FAIRFAX STREET, SUITE 900, ALEXANDRIA, VA, 22314, US

(21) Appl. No.: 10/524,418

(22) PCT Filed: Aug. 12, 2003
(86) PCT No.: PCT/GB03/03511

§ 371 Date: Feb. 14, 2005

(30) Foreign Application Priority Data
Aug. 13, 2002 (GB) GB0218797.9

(51) Int. Cl. C12Q 1/68

(52) U.S. Cl. 435/6; 422/68.1; 436/518

(57) ABSTRACT

The invention provides an analysis system for capturing target molecules in a sample. The system comprises: (a) supports with a largest dimension of 500 μm or less, wherein each support includes at least one capture analyte bound thereto, said at least one analyte being at least one capture agent exhibiting an affinity for one or more of proteins, antibodies, antibody fragments, DNA aptamers, nucleic acids, small molecules and any other molecules used to bind target molecules; and (b) an arrangement for introducing said sample into contact with said at least one analyte of at least one support in a fluid solution, such that binding of at least one target molecule with at least one analyte is indicative of the presence of said at least one target molecule. The system is distinguished in that: (c) each support comprises identification features for enabling the system to identify the support; (d) the system includes an arrangement for detecting binding of said at least one target molecule with said at least one analyte, the system thereby being capable of associating each support with its corresponding target molecule; and (e) the system further including an additional arrangement for recovering and analysing a remainder of said sample whose molecules are not susceptible to capture by said at least one analyte bound to said supports.

### FIELD OF THE INVENTION

The present invention relates to an analysis system for capturing and filtering target molecules in samples to reduce the complexity of the samples such that both captured and remaining uncaptured molecules may be characterised. Moreover, the invention further relates to a method of capturing and filtering target molecules in samples to reduce the complexity of the samples such that both captured and remaining uncaptured molecules may be characterised.

### BACKGROUND TO THE INVENTION

During recent years, there has arisen a considerable interest in techniques and associated systems for determining protein and nucleic acid characteristics of numerous types of organisms, for example, yeast, bacteria and mammals as well as cell lines. There is, for example, currently a need for massively parallel high throughput technologies for identification and characterisation of proteins (proteomics) in biotechnological, pharmaceutical, diagnostic, veterinary, petroleum, pulp and paper, food and beverage, and chemical industries.

Similarly, there has also recently arisen a considerable interest in techniques and associated systems for high throughput analysis of complex samples, for example, yeast, bacteria, mammals, cell lines, drug targets and potential therapeutics. Such analysis includes high throughput profiling of protein or gene expression providing high volumes of information about cell events. Mechanisms behind disease and the effects of therapeutics are associated with protein and genetic profiles; hence, their analysis provides information for the development of diagnostic tests and new drugs. However, attempting to simultaneously analyse all cell events is an exceedingly complicated task, therefore samples must conventionally first be simplified by, for example, fractionation into related subgroups such as mitochondria or nucleic acids.

For example, the field of proteomics, namely the simultaneous analysis of total gene expression at the protein level, has rapidly become one of the leading approaches for studying biological systems and understanding the relationship between various expressed genes and gene products. As knowledge about the human genome accumulates, there has been a parallel interest in developing techniques and associated systems for corresponding proteomes, namely the entire complement of proteins expressed by a particular cell, organism or tissue type. Particular interest has been shown in the development of techniques for determining the characteristics of proteomes associated with particular disease states or specific sets of environmental conditions.

Currently, tests for detecting protein characteristics in a sample require a large number of experimental steps. The steps include preparation of the sample by lysis, followed by two-dimensional gel electrophoresis (2D-GE), post-electrophoresis extraction of the proteins followed by mass spectrophotometry, chromatography, microarrays or additional electrophoresis methods. A core method presently used in proteomics, namely 2D-GE, is a technique capable of resolving thousands of proteins and peptides from a single complex mixture in a single experiment Proteins are first separated according to their isoelectric point, namely the pH at which their net charge is zero, and then orthogonally separated based on apparent mass using an electrophoresis step. The individual proteins are revealed as isolated spots on the gel by applying standard staining protocols. However, like many conventional methodologies, 2D-GE analysis suffers from a number of serious limitations that bring into question the utility of this procedure for adaptation to high-throughput capacity. Such serious limitations include the number of experimental steps required to identify a protein, poor reproducibility, difficulty in resolution, an inability to visualize low-abundance proteins, and the high degree of technical skill and sophisticated computational analysis to identify protein spots that are present on the gel or blot from one extract but not on the other.

Such aforementioned methods typically result in approximately half of the proteins in a given cell being characterised. In order to characterise remaining proteins, the above methods are repeated again at least once. Most researchers believe that 2D-GE, in its present format represents the most significant bottleneck to large-scale proteomics research mainly because it is possible to identify only most abundant proteins in a cell lysate from a 2D gel of a total cellular extract; for example, only 100 to 600 most abundant proteins represents only a fraction of the one billion different proteins/isoforms that may exist. Typically, when analysing a 2D gel of a total cellular extract, proteins representing only about 250 different gene products are analysed. Since protein separation in 2D gels is based on isoelectric point and molecular weight, any polypeptides with similar properties are unresolved, namely they will be on the same spot on the gel.

In the discovery of new drug targets, analyses must be expanded beyond the most abundant and best-characterised proteins of cells. The large number of these abundant proteins often causes problems during analysis. Differential fractionation of the cells normally splits the cells into components such as nucleus, cytoplasm, and mitochondria groups which are analysed using other techniques such. as chromatography and immunoprecipitation prior to applying standard 2D gels. A consecutive approach of splitting samples into components and using several different methods for analysis for the components is however time consuming and requires highly skilled technicians to perform associated experiments.

A common way to filter proteins and peptides from a cellular extract prior to their analysis on a 2D gel or on a protein array is an affinity capture assay. This assay involves using known capture molecules such as antibodies to screen a cell lysate; the antibodies bind their respective targets and the remaining corresponding supernatant can be analysed using a 2D gel procedure as described in a published article Li, J. et al. Mol Cell Proteomics 2002 February 1 (2): pp. 157-68.

There are many examples of affinity capture systems described in the prior art. For example, in a published PCT patent application no. PCT/GB01/04182, there is described Oxford Glycosciences Ltd.'s microarray affinity capture system in which antibodies are bound to a fixed array and used to bind known peptide fragments from a lysed sample.

Moreover, in a published PCT patent application no. PCT/US99/12708 from Immco Diagnostics Ltd., there is described a method for the quantitation of an analyte in a test sample using an affinity assay. The analyte is bound with a first affinity molecule to form a complex. The complex is then immobilised to a solid matrix and contacted with a labelled second affinity molecule to label immobilized complexes containing the analyte. The amount of analyte in the sample is then quantitated from the amount of label immobilized. In a PCT patent application no. PCT/US98/12843, Ciphergen ProteinChip® describes use of arrays which exhibit specific surface chemistries to affinity-capture minute quantities of proteins. Such technology requires the use of Surface Enhanced Laser Desorption/Ionisation (SELDI) to identify the captured proteins. Some common drawbacks of such techniques are induced denaturation of peptides, non-specific binding analytes and interaction of adjacent molecules on the arrays. Similar issues arise when capturing target molecules from complex mixtures of nucleic acids and small molecules such as chemical compounds that may be potential therapeutics.

A method for performing affinity assays with a retrievable support comprising a magnetic bead, which can reversibly bind to target molecule, is described in a patent no. EP0265244 by Amoco Ltd. Beads have been used to develop a quantitative antibody capture test for C-reactive protein as described in a scientific article Tarkkinen, P. et al., Clin Chem 2002 February 48 (2): pp. 269-77. Both the method and the test employ capture analytes attached to the beads for capture of the target analyte.

Isotope-coded affinity tags (ICAT) have also been developed for selective affinity capture of molecules from complex samples as described in a scientific article Turecek, F. J Mass Spectrom 2002 January 37 (1): pp. 1-14.

Affinity capture may also be used for purifying complex mixtures of nucleic acids or small molecules. In a European patent application no. EP0296557A2, there is described a method of removing undesired single stranded nucleic acids from a complex mixture of single and double stranded molecules. The capture analyte consists of single stranded nucleic acids bound to water insoluble beads.

A published U.S. Pat. No. 5,759,778 is concerned with a method for isolating and recovering target nucleic acid molecules from a library using biotinylated probes comprising a complementary sequence to the target sequence. Moreover, in international PCT patent application no. PCT/US97/02852, there is described a binding assay for detecting small molecules such as environmental contaminants, drugs of abuse, therapeutic drugs and hormones. The assay involves use of a chromatographic strip containing analyte receptors for binding target analytes.

The inventors have appreciated limitations of aforementioned methods, techniques and assays and thereby devised an analysis system that is capable of addressing these limitations.

### SUMMARY OF THE INVENTION

A first object of the invention is to provide an improved analysis system for the analysis of molecules.

A second object of the invention is to provide an analysis system to improve the efficiency of analysis of molecules in complex samples.

A third object of the invention is to provide an analysis system to improve the testing throughput of conventional sample analysis apparatus.

According to a first aspect of the invention, there is provided an analysis system as defined in the accompanying claim 1.

The system is of advantage in that it is capable of addressing at least one of the aforementioned objects of the invention.

The invention concerns a method for molecule capture from a complex mixture, where uniquely encoded supports have a capture molecule attached to a main surface thereof. A multiplexed experiment of hundreds of thousands of tests in one is possible since a large number of labelled supports and attached capture molecules can be present in the assay simultaneously. Use of such capture molecules in combination with supports allows identification and recovery of the captured molecules.

In a preferred embodiment of the invention, the primary supports are in the form of microparticles decreasing the amount of reagents used for each simultaneous testing process.

The present invention preferably incorporates in an analysis system a coded three-dimensional microparticle array for use in reversible affinity capture assays. The system comprises coded microparticles to which affinity capture analytes are attached, wherein the microparticles are in solution and/or packed into a column; such an arrangement allows for large scale multiplexing of the assays. The analytes may include, but are not limited to, antibodies, antigens, proteins, enzyme substrate, carbohydrates, peptides, affibodies™, nucleic acids, peptide nucleic acids, cell lines, chemical components, oligonucleotides, serum components, small synthesised molecules, drugs or any derivatives or fragments thereof. This invention offers the benefit of separating captured molecules from a complex sample, namely each encoded microparticle carries a different captured target, which can potentially prevent unwanted interactions between target molecules and maintains the molecules in solution to prevent denaturation. The coded microparticles enable the reversibly captured molecules to be identified, recovered and characterised without complicated analysis methods such as 2D-GE. By filtering out the target molecules, the system reduces sample complexity and hence throughput of the analysis. There are a wide range of applications for the analysis system, for example comparative analysis of proteins in cell populations and drug target screening assays.

In another preferred embodiment of the invention, the identification means comprises one or more distinguishing geometrical features, such as shape, size, barcode or dotcode, enabling identification of each support. This allows the use of well-established identification standards such as for example barcodes which give good signal to noise ratio and decrease the risk of spectral overlap and false positives.

Other preferred embodiments of the invention, comprises the use of radio frequency identification transponders (RFID) or optical identification, such as fluorescence or colour coding. The use of RFID gives an advantage of very large numbers of codes can be used and does not require visual communication between the measuring means and the identifiable support. The use of optical coding on the supports allows for combinations of wavelengths or colours not possible with standard fluorescent markers, for example FITC labelled, and allows for using low cost labelled supports.

According to a second aspect of the invention, there is provided a method as defined in the accompanying claim 12.

In the second aspect of the invention, there is provided an analysis system for detecting and quantitating molecule characteristics, which has detecting means and identifying means arranged to register two different types of signals, the first signal being associated with the detection and quantification of activated signal emitting labels and the second signal being associated with the reading of sequential identification of supports. Such plurality of different types of signal decreases the potential requirement of using advanced and costly image processing equipment.

The method is of advantage in that it is capable of addressing at least one of the aforementioned objects of the invention.

It will be appreciated that features of the invention can be combined in any combination without departing from the scope of the invention.

### DESCRIPTION OF THE DRAWINGS

Embodiments of the invention will now be described, by way of example only, with reference to the accompanying drawings wherein:

FIG. 1 is a plan view and a side view of a single support (microcarrier) comprising a sequential identification;

FIG. 2 is a schematic sectional side view of a single support (microcarrier) with analytes attached thereto;

FIG. 3 is a schematic diagram of an analysis system for an assay,

FIG. 4 is a schematic diagram of an analysis system for a capture assay with a detailed view;

FIG. 5 is a schematic diagram illustrating the elution of captured molecules through a column;

FIG. 6 is a diagram of a flow-based analysis system for analysing the supports of FIG. 4;

FIG. 7 is a schematic diagram illustrating a planar-based reader for interrogating the analysis system of FIG. 4; and

FIG. 8 is a schematic diagram illustrating an alternative flush reader for interrogating the analysis system of FIG. 4.

### DESCRIPTION OF EMBODIMENTS OF THE INVENTION

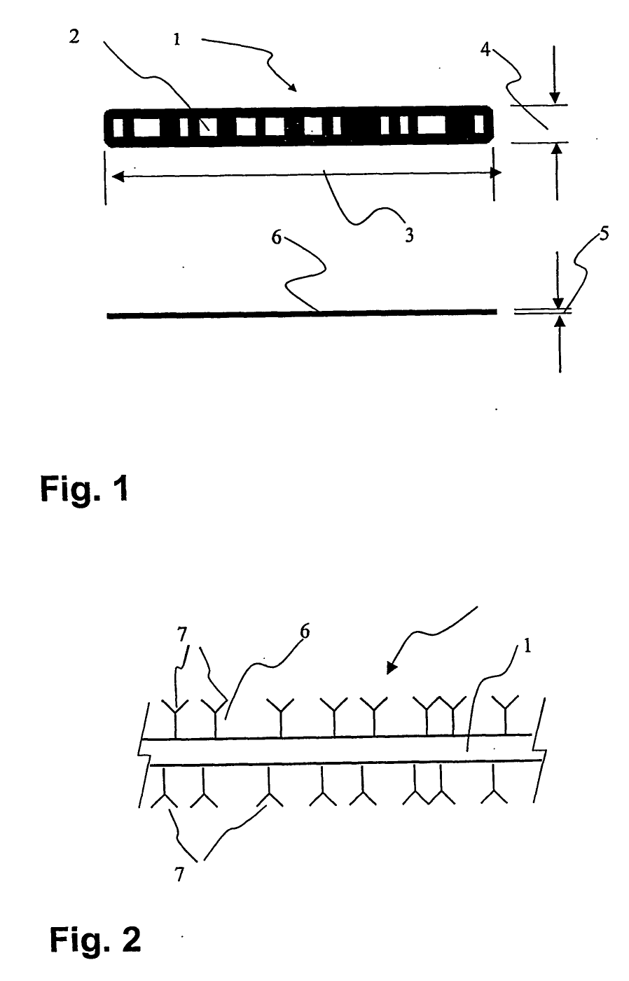

In FIG. 1, there is shown an illustration of a preferred embodiment of a support for use in an analysis system according to the invention. There is shown a single primary support 1, such a support will also be referred to as a microcarrier, microparticle or “bead” in the following description. The support 1 can be fabricated from virtually any insoluble or solid material, for example one or more of polymers, silicates, glasses, fibres, metals or metal alloys. In the preferred embodiment of the invention, the support 1 is fabricated from a metal, such as gold, silver, copper, nickel, zinc or most preferably aluminium. It is also preferable to use one or more polymers, such as polystyrenes, polyacrylates, polyamides, or polycarbonates when fabricating the support 1. The support 1 is preferably either partially or totally coated in one or more of either of the above-mentioned materials.

The support 1 incorporates an identification feature 2 that is also referred to as an identification code or tag in the following description. Examples of the identification feature 2 may be based on one or more of sequential identification, varied shape and size of the support 1, transponders (for example Radio Frequency Identification Chips, RFIDs) attached to the support 1, and fluorescent coding or different colours of the support 1. Preferably, the identification feature 2 is a sequential identification that can be in the shape of at least one (or any combination thereof) of grooves, notches, depressions, protrusions, projections, and most preferably holes. The identification feature 2 being part of the support 1 is advantageous in that there is no need to label each support 1 after manufacture. The sequential identification 2 is suitably a transmission optical barcode, which is machine readable, allowing enhanced signal to noise ratio if read in transmission or reflection. An associated sequential identification code is thereby recorded in the support 1 as a series of holes using coding schemes similar to those found on conventional bar code systems, for example as employed for labelling merchandise in commercial retailing outlets. Such a code allows the use of existing reader technology to determine the identification feature 2 of the support 1 thereby decreasing the initial investment when adopting technology according to the invention.

In the preferred embodiment, the primary support 1 is of substantially planar form with at least a principal surface 6 as illustrated in FIG. 1. The support 1 has suitably a width 4 to length 3 ratio in a range of circa 1:2 to circa 1:20, although a ratio range of circa 1:5 to circa 1:15 is especially preferred. Moreover, the support 1 has a thickness 5 that is preferably less than circa 3 μm, and more preferably less than circa 1 μm. When the thickness is less than circa 1 μm, it has been shown to provide sufficient mechanical support strength for rendering the support 1 useable in harsh experimental conditions. The largest dimension 3 of the support 1 is circa 500 μm or less, preferably circa 300 μm or less, more preferably circa 150 μm or less, most preferably circa 100 μm or less, yet more preferably circa 50 μm or less, or preferably even circa 10 μm or less in length. A preferred embodiment of the invention concerns the support 1 having a length 3 of circa 100 μm, a width 4 of circa 10 μm and a thickness 5 of circa 1 μm; such a support is capable of storing more than 100,000 different identification sequence bar codes 2.

Around 2.5 million supports similar to the support 1 may be fabricated on a single 3-inch diameter semiconductor-type wafer, for example a silicon wafer, using contemporary established manufacturing techniques. Advantageously, the shape of the support 1 is such that it optimises the number of supports 1 manufactured per wafer and also substantially optimises the number of identification codes possible on the supports 1. The support 1 utilises the benefits of a cost effective manufacturing technique with the possibility to tailor the design and identification coding as required. As described in the foregoing, the shape as well as the size of the supports 1 may be varied as appropriate using microfabrication manufacturing techniques. Non-exhaustive examples of possible shapes are, for example, circular, elliptical, elongated, square, rectangular, multi-cornered or even multi-layered supports of the same or different materials. It is also, in some applications, preferable to have the supports 1 in the size of nanoparticles with a largest dimension of circa 500 μm or less; examples of such nanoparticles comprise cylindrical nanobars. However, a lower limit to size is governed by sufficient sensitivity of the reaction kinetics being achieved.

Conventional photolithography and dry etching processes are examples of such manufacturing techniques used to manufacture and pattern a material layer to yield separate solid supports 1 with bar-coded identification 2.

A fabrication process for manufacturing a plurality of supports similar to the support 1 involves the following steps:
(1) depositing a soluble release layer onto a planar wafer;
(2) depositing a layer of support material onto the release layer remote from the wafer;
(3) defining support features, including the sequential identification feature 2, in the support material layer by way of photolithographic processes and etching processes;
(4) removing the release layer using an appropriate solvent to yield the supports released from the planar wafer; and
(5) optionally treating the support material to improve its attachment properties.

FIG. 2 provides an illustration of how capture analytes 7, such as proteins, antibodies, antibody fragments, DNA aptamers, nucleic acids, affibodies™, small molecules and any other molecules used as capture analytes 7, are attached to a section 6 of the support 1. Many methods of chemically treating or physically altering the support material may be used for the optional step (5) to facilitate the attachment of a capture analyte. Alternatively, the treatment of the support material layer, step (5), can be optionally omitted. The treatment of the supports 1 can be performed after the release from the wafer as described above or alternatively prior to the release from the wafers or earlier in the manufacturing process steps. By modifying the surface 6 of the supports 1 or the capture analytes 7, the attachment between capture analytes 7 and supports 1 is improved.

Aluminium is a preferred material for the support 1 and there are known methods of growing porous surfaces through aluminium anodisation to improve the attachment properties thereof. Likewise, processes for sealing such porous surfaces are also known. The Applicant has exploited such knowledge to develop a relatively simple process for growing an absorbing surface having accurately controlled porosity and depth. Such porous surfaces 6 are capable of achieving a mechanical binding to the capture analyte 7. Using an avidin-biotin system is another approach for improving chemical binding between the supports 1 and their associated capture analytes 7. The supports' 1 surface 6 may also be treated with a binding material such as silane and/or biotin, to further enhance attachment properties. The supports 1 preferably have silane baked onto their surfaces 6. Attaching linking molecules, for example avidin-biotin sandwich system, to the capture analytes 7 further enhances their chemical molecular attachment properties.

The enhanced attachment is preferably achieved through having covalent bonds between the attachment surface 6 of the support 1 and the capture analytes 7. The covalent bonds prevent the capture analytes 7 from being dislodged from the supports 1 and causing disturbing background noise during analysis. There is also a potential problem that loose capture analytes 7 are capable of preventing the identification of reactions that have occurred. It is found to be important to wash the active supports 1 after attaching capture analytes 7 thereto, to remove any excess such analytes 7 that could otherwise increase the noise in the experiment during analysis. Discrimination of tests using the supports 1 is thereby enhanced through a better signal-to-noise ratio.

It will be appreciated that the capture analytes 7 are not limited to those listed above and can comprise a broad range of compounds capable of being uniquely distinguished and identified. For example the capture analytes 7 may include antibodies, antigens, proteins, enzyme substrate, carbohydrates, peptides, affibodies™, nucleic acids, peptide nucleic acids, cell lines, chemical components, oligonucleotides, serum components, small synthesised molecules, drugs or any derivatives or fragments thereof. All capture analytes 7 in this broad range may be attached to supports fabricated by steps (1) to (5) above either before or after executing photolithographic operations or releasing the supports 1 from the planar substrate.

Appropriate identification of supports 1 as mentioned above concerns the importance of using a specific identification for a specific capture analyte 7. Such an arrangement also allows the use of predetermined identification codes 2 for certain capture analytes 7 but also allows for matching of identification codes 2 and capture analytes 7 as desired when designing an experiment.

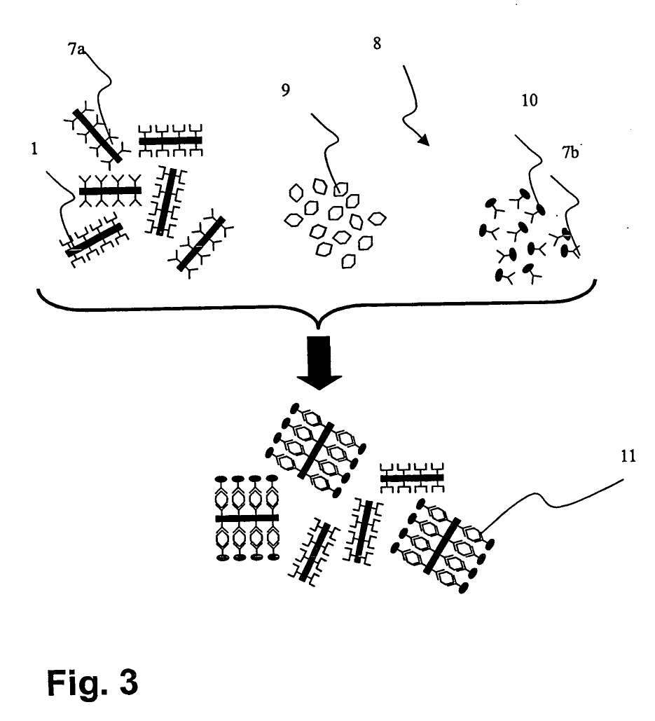

FIG. 3 shows a general method 8 whereby:
(1) a sample containing target molecules 9 is put in contact with the capture analytes 7a bound to the supports 1; and
(2) signal emitting labels 10 are bound to capture analytes 7b.

Each support 1 with its corresponding specific sequential identification code 2 has associated therewith a unique capture agent capture analyte 7a, for example a peptide or antibody associated therewith, which binds to and/or interacts with a specific target molecule 9. The signal emitting labels 10 are for example fluorescent labels. Only supports 1 with capture analytes 7a that have bound to the target molecule 9 detected will bind the signal emitting labels 10 and thereby fluoresce from their emitting labels 11. The result of the test is measured by the degree of fluorescence of different types of supports 1 with associated bound molecules. The fluorescent intensity of the bound signal emitting labels 11 quantifies the level of detected target molecules 9. Experiments where a binary yes/no reaction indication is preferred only require determination whether or not the supports 1 in the method 8 are sufficiently fluorescent relative to a predetermined fluorescence level.

Alternatively, a test sample containing target molecules is attached to a solid support such as a microtitre plate or tube. A mixture of supports 1 with bound capture analytes 7 is added. Each support 1 with its corresponding specific sequential identification features 2 has associated therewith a unique capture analyte 7, for example a peptide or antibody associated therewith, which binds to and/or interacts with a specific target molecule 9. The capture analytes 7 bind to their respective target molecules and the unbound supports 1 are washed away. The bound supports 1 are dissociated from the test sample and read by counting the number of each support 1 type with its corresponding specific sequential identification features 2 which is proportional or inversely proportional to the amount of target molecules in the test sample. In such a method, signal emitting labels 10 are not used.

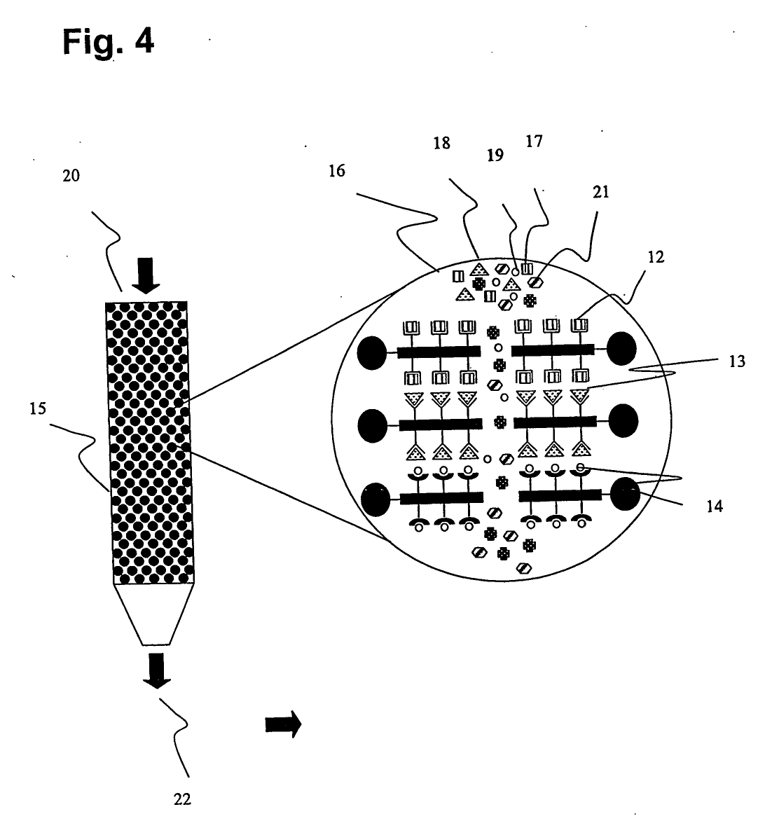

In FIG. 4, there is shown a schematic diagram of a first step of an affinity capture assay. In this example, a panel of 3 different capture analytes 12, 13, 14 have been bound to supports 1 with 3 different sequential identification 2 codes. The capture analytes 12, 13, 14 bound to the supports 1 are suspended in liquid and packed into a column 15 made of plastic or glass. The sample 16 containing the target molecules 17, 18, 19 is introduced to the top of the column 20 and moves through the column. The target molecules 17, 18, 19 are captured by their respective affinity capture analytes 12, 13, 14, while molecules 21 in the sample 16 for which there is no capture analyte 7 present will pass through the column and be collected as an eluent 22 that can be subjected to further analysis.

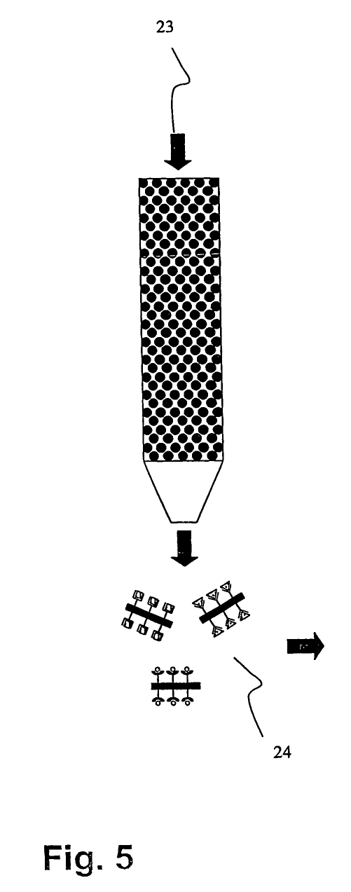

In FIG. 5, there is illustrated shown a second next step performed in the affinity capture assay. An elution buffer 23 is added to the top of the column 20. This elutes 24 the capture analytes 12, 13, 14 with their bound target molecules 17, 18, 19 from the column for further analysis. Furthermore, as the capture assay may be reversible, the target molecules 17, 18, 19 could be removed from the capture analytes 12, 13, 14 for further analysis such as quantitation. The sequential identification 2 codes on the supports 1 allow for identification and recovery of specific target molecules.

The number of different types of supports 1 used for the affinity capture assay of FIGS. 4 and 5 is dependant on the test throughput required, but could be hundreds, thousands or even millions of analytes. The number of the same types of supports 1 employed is dependent, amongst other things, on the quality of statistical analysis desired and the ease of analysis desired. Signal emitting labels 10 are also added to the affinity capture assay. These signal emitting labels 10 are used to indicate interaction, namely bonding, between the capture analytes 7 on the supports 1 and the target molecules 9 sought in the analysed sample 16. There are many different ways of adding the signal emitting labels 10 to the affinity capture assay. They can, for example, be added to the column 15 separately, be attached to the target molecule 9 to be analysed prior to the sample 16 being added to the column 15, or be attached to the capture analyte 7 before or after their attachment to the supports 1. There are also many different ways for the signal emitting labels 10 to indicate that interaction between the capture analytes 7 and the target molecule 9 in the analysed sample 16. One such way is for a signal, such as fluorescence or light of other wavelength (colour), to be activated by the signal emitting label 10 if there is interaction between capture analyte 7, a matching target molecule 9 and the signal emitting label 10. Alternatively the signal emitting labels 10 are activated before any interaction with the target molecule 9. When there is an interaction between the capture analyte 7 and the target molecule 9, the active signal emitting label 10 is released from the other molecules deactivating its signal. This would result in a detection that is opposite to the ones discussed previously, namely the absence of a signal indicates that a reaction has occurred on a support in, for example, a yes/no experiment. Similarly, a decrease in the fluorescent signal from the emitting labels 11 can be an indicator of the amount of target molecule 9 present in the analysed sample 16 introduced into the column 15.

Applications for the affinity capture assay include protein profiling of a cell, tissue, organ or whole organism or a cellular extract, lysate or protein fraction derived therefrom. Such an assay can also be used for determining the epitope profile of cells, tissues, organs and whole organisms and cellular extracts, lysates or protein fractions derived therefrom Such applications are relevant for analysis of drug targets, libraries of potential therapeutic agents and for diagnostics. Since the system of the invention reduces the complexity of samples by first filtering out known target molecules 9 with their respective capture analytes 7, it therefore enriches the sample for low quantity molecules whose identify and function may then be more easily elucidated.

By way of example, a sample, for instance derived from a cell culture, is first lysed to release all the proteins and peptides in solution, namely >10,000 proteins per cell. The lysate is introduced to the column shown in FIG. 4, on which there are supports 1 containing bound capture analytes 7. The capture analytes 7 were previously selected to capture, for example, specific peptide isoforms. The resulting sample eluent then contains only those molecules not captured by the capture analyte 7, namely the sample is enriched for uncharacterised molecules and the experiment can now focus on characterising the unknowns. A further advantage is that by reducing the total number of input molecules to the experiment, researcher using the system of the invention are less likely to detect those molecules that would overlap in analysis, for example peptides which electrophorese to the same spot on 2D-GE

The large number of supports 1 with sequential identification codes 2 available means that as new target molecules 9 are identified and capture analytes 7 developed for them, they can be added to the affinity capture assay, therefore providing a means for enriching the samples for low abundance molecules.

Reader systems for use with the reversible affinity capture assay supports will now be described. The Applicant has developed two classes of reader systems. These are based on flow cells for handling the supports 1, and on planar imaging of plated-out supports 1.

A flow-based reader system, similar in construction to a flow cytometer, can be used to draw through thousands of supports 1 per second, thereby reading simultaneously the sequential identification code 2 of each support 1 and the results of its associated test result. The test result is measured as a yes/no binary result or by the degree of fluorescence 11.

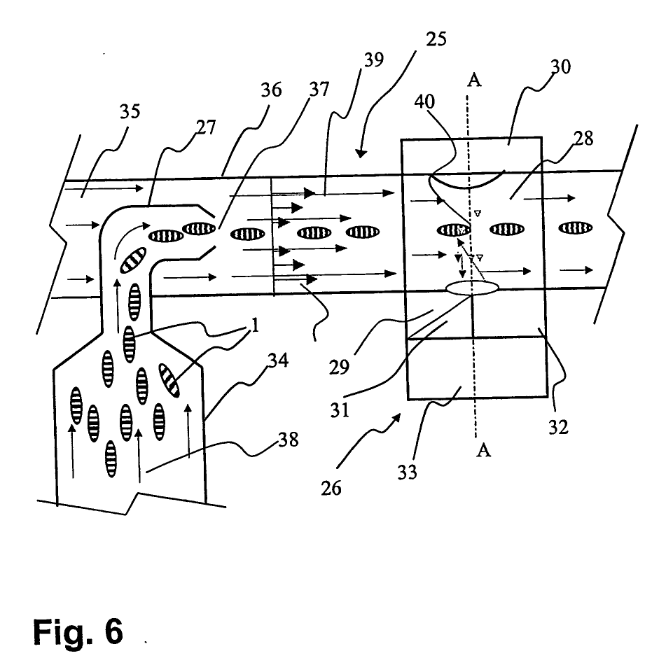

In FIG. 6, the flow-based reader system is shown indicated generally by 25. At a downstream end, the reader system 25 comprises a measuring unit indicated by 26 for reading supports 1 conveyed in operation in fluid flow from an injecting nozzle 27 at an upstream end to the measuring apparatus 26 at the downstream end. The apparatus 26 includes a reading zone 28, a reader unit 29, a light source 30, a detector unit 31, a signal emitting unit 32 and a processing unit 33.

A sample 38, for example a liquid comprising a plurality of the supports 1 dispersed therein, is introduced into the focussing zone 34. Moreover, a flow of carrier fluid 35 is generated along a tube 36 in a direction from the upstream end towards the downstream end. Preferably, the carrier fluid 35 flowing in operation along the tube 36 is a liquid. Alternatively, the fluid 35 can be a gas at reduced pressure relative to the nozzle 27 so that liquid bearing the supports 1 to an exit aperture 37 is vaporised at the aperture 37, thereby assisting to launch supports 1 into the tube 36. Whereas it is easier to establish a laminar flow regime within the tube 36 when fluid flowing therethrough is a liquid, gas flow through the tube 36 potentially offers extremely fast support 1 throughput and associated interrogation in the reading zone 28.

The reader 25 is designed to induce the supports 1 to flow along a central region of a tube 36 through the defined interrogation zone 28. By utilizing an accelerated sheath fluid 35 configuration and the injecting nozzle 27, the supports 1 injected into the central region of the tube 36 are subjected to a hydrodynamic focusing effect 39 causing all the supports 1 to align lengthwise, namely axially, and to pass through a well-defined focal point 40 in the interrogation zone 28 downstream from the exit aperture 37. The distance between the exit aperture 37 and the interrogation zone 28 must be sufficiently long to dissipate any turbulence caused by the injection nozzle 27. This sufficient length allows for a substantially laminar flow of the reading fluid 35 and hence provides the supports 1 with a non-oscillating movement past the focal point 40. If required, the nozzle 27 can be provided with a radially symmetrical arrangement of feed tubelets from the focussing zone 34 so as to obtain a more symmetrical velocity profile within the tube 36. In an interface surface region in close proximity to the peripheral surfaces of the tube 36, fluid velocity progressively reduces to substantially zero at the interior surface of the tube 36.

The supports 1 are ejected from the exit aperture 37 and are swept in the flow 35 along the tube 36 into the reading zone 28 and eventually therepast. When one or more of the supports 1 enter the reading zone 28, light from the source 30 illuminates the one or more supports 1 at the focal point 40 so that they appear in silhouette view at the reader unit 29. The reader unit 29 generates a corresponding silhouette signal that is communicated to the processing unit 33 for subsequent image processing to determine the sequential identification 2 of the supports 1. Preferably, the light source 30 emits light in a plane A-A that is substantially perpendicular to the samples' flow 35 direction and from two different radial directions, the radial directions preferably having a mutual angle separation, for example with a mutual angular separation of circa 45° separation. Such an arrangement of support 1 illumination in the focal point 40 enables the supports 1 to be identified irrespectively of their rotational position along their longitudinal axis.

For each support 1 transported through the zone 34, the processing unit 33 is programmed to determine the sequential identification 2 of the support 1 with its corresponding magnitude of fluorescence. The reader unit 29, located substantially at an opposite side of the interrogation zone 28 relative to the light source 30, reads the light that passes through one or more supports 1 at the focal point 40. The detector unit 32 detects any fluorescence occurring in the zone 28 and generates a corresponding fluorescence signal that is subsequently received by the processing unit 33. The detector unit 31 measures the magnitude of the intensity of the fluorescent signal 11 that is given off by the activated signal labels 10 on the supports 1. This intensity indicates the degree of reaction that can be extrapolated to determine the relative amount of reactive target molecule 9 present in the sample. Moreover, the processing unit 33 is also connected to an associated database relating the sequential identification 2 with a test provided by its associated capture analytes 7.

Examples of successful experiments of sorting supports I with fluorescent signal indicating successful capture of wanted molecule has been performed on the Union Biometrica, COPAS™ flow cytometer at a rate of 20 supports 1 per second and sorting one support 1 into a well of a microtitre plate with 96-well format. These results in the COPAS™ can be qualitative or quantitative depending on the experiment requirements. Other flow cytometers also successfully used for measuring and sorting out positive response supports 1 are the MoFlo™ from DakoCytomation and the FACScan™ from Becton Dickenson.

A feature in the form of a marking at one end of each support 1 is used to indicate to the reader unit 29 how to interpret the read information. This allows the support 1 to be read from either direction along its longitudinal axis. The marking is also susceptible to being used to increase the number of possible sequential identification codes on a support 1 to be greatly in excess of 100,000. For example, employing four different markings on separate sets of supports 1 is capable of increasing the number of identification combinations of supports to about 400,000. An alternative feature to indicate how information codes are to be read is to start each block with 0's and end the blocks with 1's, or vice versa. Further alternatives of these features are preferably error correction data, for parity bit checks and/or forward error correction, thereby improving testing reliability.

As an alternative to the flow-based reader system of FIG. 6, a planar reader system can be employed, wherein:
(a) the supports 1 are plated out onto a planar substrate; and then
(b) fluorescence microscopy and associated image processing are employed to read the bar codes of the supports and the results of their associated tests.

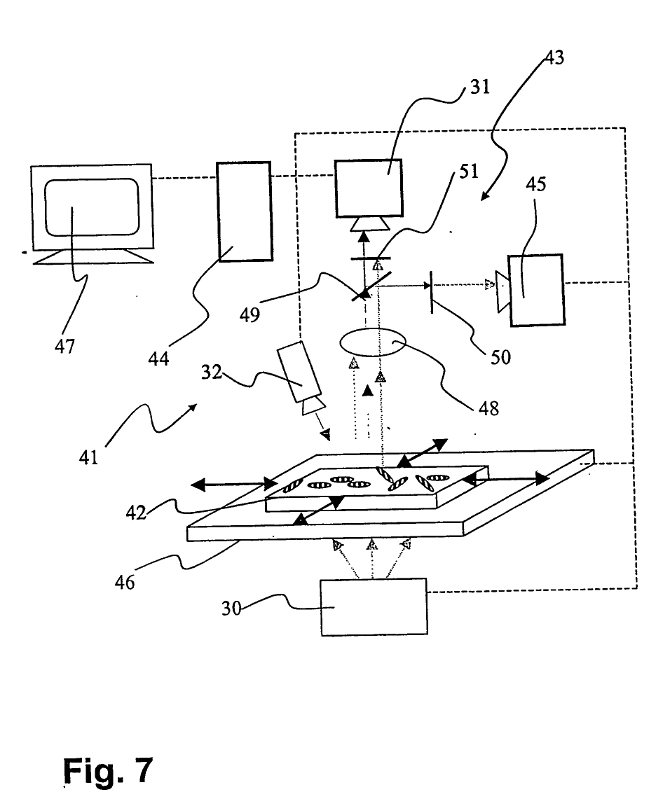

In FIG. 7, there is shown a planar reader system indicated generally by 41. After the capture assay has been completed as described with reference to FIG. 6, the supports 1 are deposited on the planar substrate 42. Preferably, the planar substrate 42 is fabricated from a polymer, glass or silicon-based material, for example a microscope slide. A planar measuring unit 43 arranged to perform conventional fluorescence microscopy is used to analyse the support-plated substrate 42 systematically, measuring the level of fluorescence of emitting labels 11 thereon and also the sequential identification 2 of the different supports 1 of the support-loaded substrate 42. Normally, all the supports 1 on the loaded substrate 42 are analysed to verify the total quality of the experiment. In cases where it is desirable to save time and/or to increase processing capacity, software executing in a processing unit 44 of the reader system 41 can preferably be configured to analyse only the supports 1 whose emitting labels 11 fluoresce, for example by virtue of their fluorescent signal labels 10, indicating that an interaction with the target molecule 9 has occurred. The analysis of the loaded substrate 42 using the planar measuring unit 43 is a very cost effective, easy to perform and suitable way to multiply the analysing capacity for low to medium sample numbers in the range of, for example, single figures to a few thousand supports on each substrate 42.

The planar measuring unit's 43 reader unit 45 for image-processing is used to capture digital images of each field of the substrate 42 to which supports 1 have become affixed. Digital images thereby obtained correspond to light transmitted through the substrate 42 and its associated base plate 46 and then through the supports 1 rendering the supports 1 in silhouette view; such silhouette images of the supports 1 are analysed by the reader unit 45 in combination with a processing unit 44. The sequential identification 2 of the supports 1 may also be read by reflected light. The sequential identification 2, for example a bar-code, associated with each support 1 is hence identified from its transmitted or reflected light profile by the reader unit 45. The signal emitting unit 32 generates a fluorescent signal, which signal makes the signal emitting labels 11 on supports 1 that have interacted with the target molecules 9 fluoresce. A detector unit 31 detects the magnitude of fluorescence from activated supports 1 to identify the degree of reaction. The fluorescent signal integrated over activated supports' 1 surface 6 is recorded in association with the identification bar-code 2 to construct data sets susceptible to statistical analysis.

The processing unit 44 is connected to the light source 30, the signal unit 32, the reader unit 45, and the detector unit 31 and to a display 47. Moreover, the processing unit 44 comprises a control system for controlling the light source 30 and the signal unit 32. The light silhouette or reflected light and fluorescent signals from the supports 1 pass via an optical assembly 48, for example an assembly comprising one or more lenses and/or one or more mirrors or electrochemical shutters and filter wheels, towards the detector unit 31 and reader unit 45. By way of example, a mirror assembly as shown can be employed in the reader system 41. A mirror 49 is used to divide the optical signals into two paths and optical filters 50, 51 for filtering out unwanted optical signals based on their wavelength. Alternatively, the light source 30 and signal unit 32 can be turned on and off at intervals, for example mutually alternately. Signals are received from the reader unit 45 and detector unit 31, these signals being processed and corresponding statistical analysis results presented on a display 47. Similar numbers of each type of supports 1 are required to give optimal statistical analysis of experiments. Such statistical analysis is well known in the art.

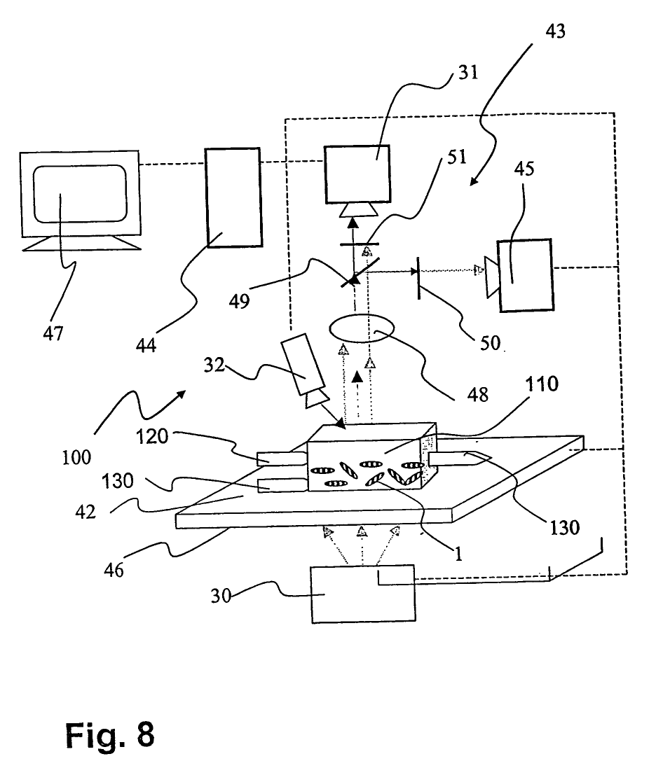

In FIG. 8, there is shown the flush cell reader system and indicated generally by 100. The flush system 100 is configured in a similar manner to the planar system 41 but employs a flushing action to introduce supports 1 to be read. After the aforementioned capture assay has been completed, the supports 1 are flushed into the reader cell 110 via a sample inflow tube 120. Preferably, the reader cell 42 is fabricated from a clear polymer, glass or silicon-based materials, for example Perspex. The measuring unit 43 is arranged to perform conventional fluorescence microscopy and is used in operation to analyse the supports 1 that have settled onto a base of the reader cell 110, thereby measuring the level of fluorescence of supports 1 thereon, and also the corresponding sequential identification 2 of the supports 1 thereon. Normally, all the supports 1 on the loaded reader cell 110 are analysed to verify the total quality of the experiment. In cases where there could be an interest in saving time and/or increasing processing capacity, the software of the processing unit 44 is preferably configurable to analyse only the supports 1 that emit a fluorescent signal, namely their including fluorescent signal labels 10 fluoresce, indicating that an interaction with the target molecule 9 has occurred. The analysis of the loaded reader cell 110 using the planar measuring unit 43 is a very cost effective, easy to perform and suitable way to multiply the analysing capacity for low to medium sample numbers in the range of, for example, single figures to a few thousand supports on each reader cell 110.

The planar measuring unit's 43 reader unit 45 for image-processing is used to capture digital images of each field of the reader cell 110 to which supports 1 have settled. Digital images thereby obtained correspond to light transmitted through the reader cell 110 and its base plate 46 and then through the supports 1 rendering the supports 1 in silhouette view; such silhouette images of the supports 1 are analysed by the reader unit 45 in combination with a processing unit 44. The sequential identification 2 of the supports 1 may also be read by reflected light. The sequential identification 2, for example a bar-code, associated with each support 1 is hence identified from its transmitted or reflected light profile by the reader unit 45. The signal emitting unit 32 generates a fluorescent signal, which signal makes the signal emitting labels 11 on supports 1 that have interacted with the target molecules 9 fluorescence. A detector unit 31 detects the magnitude of fluorescence 11 from activated supports 1 to identify the degree of reaction. The fluorescent signal integrated over activated supports' 1 surface 6 is recorded in association with the identification bar code 2 to construct data sets susceptible to statistical analysis.

The processing unit 44 is connected to the light source 30, the signal unit 32, the reader unit 45, and the detector unit 31 and to a display 47. Moreover, the processing unit 44 comprises a control system for controlling the light source 30 and the signal unit 32. The light silhouette or reflected light and fluorescent signals from the supports 1 pass via an optical assembly 48, for example an assembly comprising one or more lenses and/or one or more mirrors or electrochemical shutters and filter wheels, towards the detector unit 31 and reader unit 45.

By way of example, a mirror assembly is shown. A mirror 49 is used to divide the optical signals into two paths and optical filters 50, 51 are used to filter out unwanted optical signals based on their wavelength. Alternatively, the light source 30 and signal unit 32 can be turned on and off at intervals, for example mutually alternately. Signals are received from the reader unit 45 and detector unit 31, which are processed and corresponding statistical analysis results presented on a display 47. Similar numbers of each type of supports 1 are required to give optimal statistical analysis of experiments. Such statistical analysis is well known in the art.

Once the supports 1 have been identified by the system, buffer is flushed through the reader cell 110 via a buffer inflow tube 130 and the read supports 1 are washed from the reader cell 110 via an outlet tube 140. The next sample of supports to be read are then introduced via the sample inflow tube 120.

It will be appreciated that modifications can be made to embodiments of the invention described in the foregoing without departing from the scope of the invention as defined by the appended claims.

1. An analysis system for capturing target molecules in a sample, the system comprising:
(a) supports with a largest dimension of 500 μm or less, wherein each support includes at least one capture analyte bound thereto, said at least one analyte being at least one capture agent exhibiting an affinity for one or more of proteins, antibodies, antibody fragments, DNA aptamers, nucleic acids, small molecules and any other molecules used to bind target molecules;
(b) engaging means for introducing said sample into contact with said at least one analyte of at least one support in a fluid solution, such that binding of at least one target molecule with at least one analyte is indicative of the presence of said at least one target molecule;
characterised in that:
(c) each support comprises identifying means for enabling the system to identify the support;
(d) the system includes interrogating means for detecting binding of said at least one target molecule with said at least one analyte, the system thereby being capable of associating each support with its corresponding target molecule; and
(e) the system further including analysis means for recovering and analysing a remainder of said sample whose molecules are not susceptible to capture by said at least one analyte bound to said supports.

2. A system according to claim 1, wherein at least one target molecule captured onto its corresponding at least one analyte is reversibly bound thereto such that said at least one reversibly bound molecule is susceptible to being recovered, characterised and quantitated using said interrogating means.

3. A system according to claim 1, wherein the amount of target molecule present in the sample is quantitable from the amount thereof bound to said at least one capture analyte.

4. A system according to claim 1, wherein the analysis means for analysing the remainder of the sample includes one or more of the following for performing such analysis: microarrays, mass spectrophotometry, 2D-GE, chromatography, sequencing, flow cytometry and immunoprecipitation.

5. A system according to claim 1, wherein the largest dimension of the support is less than 300 μm.

6. A system according to claim 1, wherein the largest dimension of the support is less than 150 μm.

7. A system according to claim 1, wherein the largest dimension of the support is less than 50 μm.

8. A system according to claim 1, wherein the identifying means comprises one or more of distinguishing geometrical features, such as shape, size, barcode or dotcode, enabling identification of each support.

9. A system according to claim 1, wherein at least one of the identification means is a radio frequency identification transponder (RFID).

10. A system according to claim 1, wherein at least one of the identification means is an optical identification, such as fluorescence or colour based.

11. A system according to claim 1, wherein the fluid solution is a liquid.

12. A method of capturing and filtering target molecules in a sample, the method including the steps of:
(a) providing supports with a largest dimension of 500 μm or less, wherein each support includes at least one capture analyte bound thereto, said at least one analyte being at least one capture agent exhibiting an affinity for one or more of proteins, antibodies, antibody fragments, DNA aptamers, nucleic acids, small molecules and any other molecules used to bind target molecules;
(b) introducing said sample into contact with said at least one analyte of at least one support in a fluid solution, such that binding of at least one target molecule with at least one analyte is indicative of the presence of said at least one target molecule;
characterised in that the method further comprises the step of:
(c) providing each support with identifying means for enabling identification of the support;
(d) detecting binding of said at least one target molecule with said at least one analyte, thereby associating each support with its corresponding target molecule; and
(e) recovering and analysing a remainder of said sample whose molecules are not susceptible to capture by said at least one analyte bound to said supports.

13. A method according to claim 12, wherein at least one target molecule captured onto its corresponding at least one analyte is reversibly bound thereto such that said at least one reversibly bound molecule is susceptible to being recovered, characterised and quantitated using interrogating means.

14. A method according to claim 12, wherein the amount of target molecule present in the sample is quantitable from the amount thereof bound to said at least one capture analyte.

15. A method according to claim 12, wherein in step (e) the remainder of the sample is analysed using one or more of the following: microarrays, mass spectrophoto,etry, 2D-GE, chromatography, sequencing, flow cytometry and immunoprecipitation.

16. A method according to claim 12, wherein the largest dimension of the support is less than 300 μm.

17. A method according to claim 12, wherein the largest dimension of the support is less than 150 μm.

18. A method according to claim 12, wherein the largest dimension of the support is less than 50 μm.

19. A method according to claim 12, wherein the identifying means comprises one or more of distinguishing geometrical features, such as shape, size, barcode or dotcode, enabling identification of each support.

20. A method according to claim 12, wherein at least one of the identifying means is a radio frequency identification transponder (RFID).

21. A method according to claim 12, wherein at least one of the identifying means is an optical identification, such as fluorescence or colour based.

22. A method according to claim 12, wherein the fluid solution is a liquid.

23. (canceled)

24. (canceled)
